(19) United States
(12) Patent Application Publication
(10) Pub. No.: US 2025/0037852 A1
(43) Pub. Date: Jan. 30, 2025

(54) IMAGING PROTOCOL CONFLICT MANAGEMENT SYSTEMS AND METHODS

(71) Applicant: GE Precision Healthcare LLC, Waukesha, WI (US)

(72) Inventors: Sushmita Kari, Bangalore (IN); Vishak KC, Bangalore (IN); Durga Phani Kanth Pilli, Bangalore (IN); Mohamed Ali, Bangalore (IN); Vaibhav Misra, Bangalore (IN)

(21) Appl. No.: 18/713,131

(22) PCT Filed: Nov. 23, 2022
(86) PCT No.: PCT/US2022/050918

§ 371 Date: May 23, 2024

(30) Foreign Application Priority Data
Nov. 24, 2021 (IN) 202141054334

(51) Int. Cl. G16H 40/67

(52) U.S. Cl. CPC G16H 40/67

(57) ABSTRACT

Imaging protocol conflict management systems and methods (e.g., using a computerized tool) are enabled. For example, a system can comprise a memory that stores computer executable components, and a processor that executes the computer executable components stored in the memory, wherein the computer executable components comprise a conflict component that, according to a defined conflict criterion and based on a comparison of one or more medical device protocols associated with a medical device and one or more library protocols associated with a protocol library, determines a protocol conflict, and an output component that renders an output representative of the protocol conflict.

### CROSS-REFERENCE TO RELATED APPLICATIONS

The subject patent application claims priority to India Provisional Application No. 20/214,1054334, filed Nov. 24, 2021, and entitled “IMAGING PROTOCOL CONFLICT MANAGEMENT SYSTEM,” the entirety of which priority application is hereby incorporated by reference herein.

### TECHNICAL FIELD

The disclosed subject matter relates to medical imaging protocols and, more particularly, to systems and methods for conflict management of imaging protocols between imaging devices in a hospital network.

### BACKGROUND

Medical imaging devices (e.g., magnetic resonance (MR) scanner, computed tomography (CT) scanner, x-ray acquisition system, positron emission tomography (PET) scanner, nuclear medicine (NM) scanner, etc.) use imaging procedures to obtain image data of a target, such as a patient. An imaging procedure is associated with one or more imaging protocols that define image acquisition and/or processing actions or elements, such as one or more imaging parameters, one or more scanning planes in which image(s) are to be captured, and so on. For example, an imaging protocol may include parameters or specifications for an imaging device to utilize, such as tube current, tube voltage, filter usage, filter type, scan speed, etc. An imaging protocol may define a scanning plane for the associated imaging procedure, specify position and orientation of anatomical structure(s) or region(s) of interest in the patient, etc. An imaging protocol may further specify limits and/or other guidance on image noise, spatial resolution, image texture including edge sharpness, artifacts, or radiation dose.

A medical imaging device or system maintains a protocol database which stores various imaging procedures and/or protocols for the device to use according to one or more scenarios, reasons for examination, etc. The scenarios for examination may include patient size, anatomy type (e.g., heart, lung, kidney, brain, etc.), position, task, etc. For example, imaging protocols can be constructed for particular clinical tasks. A task function such as tumor detection, tumor sizing, vessel sizing, etc., can be incorporated into an objective function to determine a dose distribution for a given task and to find a minimum possible dose for a given performance level. During protocol development, results from similar clinical tasks (e.g., tuning for a given anatomical location, etc.) can be used to inform initial parameter selection for another clinical task (e.g., bone imaging in the wrist may be used to inform the initial selection of parameters for bone imaging in the ankle, etc.).

Additionally, protocols for similar scenarios and tasks may vary on different brands/models of imaging devices. As an example, a protocol for a liver scan by imaging scanner A indicates a 120 kV tube current at 300 mA for 1 second. Scanner B of another model can rotate faster and uses a higher tube current to generate the same signal with a protocol of 120 kV at 400 mA for 0.75 second. As another example, a protocol for pediatric abdomen scan by scanner A indicates 80 kV, 200 mA, a helical pitch of 1, etc. Scanner B has a wider scan coverage such that a helical pitch can be translated to a single axial acquisition and thus uses a protocol of 70 kV, 300 mA, and axial at wide coverage. As another example, an imaging protocol for scanner A includes a first priority indicating a desired limit of radiation dose level and a second priority indicating a reduction of motion artifacts by using 80 kV at 200 mA for pediatric abdomen scan. If scanner B has lower kV capabilities, the protocol for scanner B may be adjusted to 70 kV at 300 mA. As another example, scanner A has a protocol for an inner car scan which indicates 120 kV, 200 mA, and a bone kernel filter. Scanner B has a different kernel filter that can improve bone images compared to the bone kernel filter of Scanner A, but impacts the amount of signal that is required. Therefore, the impact may be accounted for such that the scanner B protocol includes 120 kV, 300 mA, and a “bone plus” kernel.

Imaging procedure and associated imaging protocol(s) can be visualized via a graphical user interface (GUI) for a user (e.g., radiologist, technician, clinical specialist) to select. For example, an interactive user interface can include menu and control options to allow the user to select and configure an imaging protocol. For an X-ray imaging protocol for example, the interface allows the user to select an acquisition trajectory, manage radiation dose in real-time, control tube angular orientation, tube tilt, tube position, table motion and/or orientation and other parameters for imaging during reference and/or tomosynthesis scans. When the user selects the imaging protocol via the interface, an imaging procedure associated with the imaging protocol will be performed.

For an organization (e.g., hospital, clinic, etc.) that has a large fleet of imaging devices at various facilities, managing protocols for the devices can be very costly and time consuming. In some conventional systems, protocols are transferred between medical imaging devices using USB flash drives, which can lead to inconsistent protocols used within facilities or across different facilities. In others, protocols are entirely manually managed. Thus, exam quality may be inconsistent due to inconsistent protocols used, which may put patient safety and outcome at risk. Compliance with regulations and accreditation requirements may be challenging due to variability in dose, exam duration, and diagnostics quality. In addition, protocols need to be reviewed and kept current all the time. However, modification of protocols may be inefficient because protocols are modified per exam, which results in loss of productivity and revenue.

The above-described background relating to medical imaging and medical imaging protocols is merely intended to provide a contextual overview of some current issues and is not intended to be exhaustive. Other contextual information may become further apparent upon review of the following detailed description.

### BRIEF DESCRIPTION OF DRAWINGS

FIG. 1 is a block diagram of an exemplary system in accordance with one or more embodiments described herein.

FIG. 2 is a block diagram of an exemplary medical device in accordance with one or more embodiments described herein.

FIG. 3 illustrates an exemplary conflict resolution window of a graphical user interface in accordance with one or more embodiments described herein.

FIG. 4 illustrates an exemplary conflict resolution window of a graphical user interface in accordance with one or more embodiments described herein.

FIG. 5 illustrates an exemplary conflict resolution window of a graphical user interface in accordance with one or more embodiments described herein.

FIG. 6 illustrates an exemplary conflict resolution window of a graphical user interface in accordance with one or more embodiments described herein.

FIG. 7 illustrates an exemplary conflict resolution window of a graphical user interface in accordance with one or more embodiments described herein.

FIG. 8 is a flowchart of a process associated with imaging protocol conflict management in accordance with one or more embodiments described herein.

FIG. 9 is a block flow diagram for a process associated with imaging protocol conflict management in accordance with one or more embodiments described herein.

FIG. 10 is an example, non-limiting computing environment in which one or more embodiments described herein can be implemented.

FIG. 11 is an example, non-limiting networking environment in which one or more embodiments described herein can be implemented.

### DETAILED DESCRIPTION

The subject disclosure is now described with reference to the drawings, wherein like reference numerals are used to refer to like elements throughout. In the following description, for purposes of explanation, numerous specific details are set forth in order to provide a thorough understanding of the subject disclosure. It may be evident, however, that the subject disclosure may be practiced without these specific details. In other instances, well-known structures and devices are shown in block diagram form in order to facilitate describing the subject disclosure.

As alluded to above, imaging protocol conflict management can be improved in various ways, and various embodiments are described herein to this end and/or other ends.

According to an embodiment, a system can comprise a memory that stores computer executable components, and a processor that executes the computer executable components stored in the memory. The computer executable components can comprise a conflict component that, according to a defined conflict criterion and based on a comparison of one or more medical device protocols associated with a medical device and one or more library protocols associated with a protocol library, determines a protocol conflict, and an output component that renders an output representative of the protocol conflict.

In another embodiment, a method can comprise, according to a defined conflict criterion and based on a comparison of one or more medical device protocols associated with a medical device and one or more library protocols associated with a protocol library, determining, by a system comprising a processor, a protocol conflict, and generating, by the system, an output representative of the protocol conflict.

According to yet another embodiment, a non-transitory machine-readable storage medium can comprise executable instructions that, when executed by a processor, facilitate performance of operations, comprising, according to a defined conflict criterion and based on a comparison of one or more medical device protocols associated with a medical device and one or more library protocols associated with a protocol library, determining a protocol conflict, and rendering an output representative of the protocol conflict via a user interface of the medical device 126.

It should be appreciated that additional manifestations, configurations, implementations, protocols, etc. can be utilized in connection with the following components described herein or different/additional components as would be appreciated by one skilled in the art.

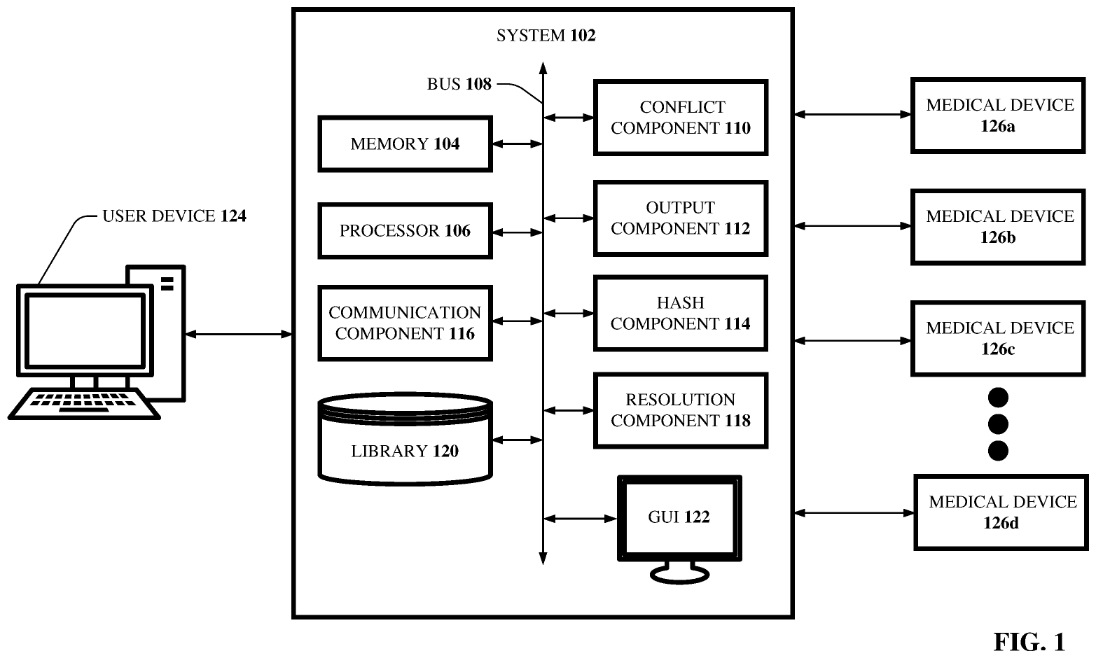

Turning now to FIG. 1, there is illustrated an example, non-limiting system 102 in accordance with one or more embodiments herein. System 102 can comprise a computerized tool, which can be configured to perform various operations relating to imaging protocol conflict management. The system 102 can comprise a system that facilitates management and/or standardization of medical imaging protocols in a hospital system or another medical setting. The system 102 can comprise one or more of a variety of components, such as memory 104, processor 106, bus 108, conflict component 110, output component 112, hash component 114, communication component 116, resolution component 118, protocol library 120, and/or graphical user interface (GUI) 122. The system 102 can be communicatively coupled to, or can further comprise, user device 124, medical device 126a, medical device 126b, medical device 126c, and/or medical device 126d. In various embodiments, one or more of the memory 104, processor 106, bus 108, conflict component 110, output component 112, hash component 114, communication component 116, resolution component 118, protocol library 120, GUI 122, user device 124, medical device 126a, medical device 126b, medical device 126c, and/or medical device 126d can be communicatively or operably coupled (e.g., over a bus or wireless network) to one another to perform one or more functions of the system 102. It is noted that the medical device 126 can comprise a magnetic resonance (MR) scanner, computed tomography (CT) scanner, x-ray imaging system, positron emission tomography (PET) scanner, nuclear medicine (NM) scanner, single-photon emission computed tomography (SPECT) scanner, and combinations thereof (e.g., multi-modality imaging systems, such as PET/CT, PET/MR or SPECT/CT imaging systems), or another suitable medical imaging or diagnostic device. In various embodiments, the GUI 122 can comprise one or more input devices (e.g., pointing device such as a mouse, keyboard, touch screen) and/or one or more output devices (e.g., display screen) in order to enable user interaction with the system 102.

According to an embodiment, the conflict component 110 can, according to a defined conflict criterion or criteria (later discussed in greater detail) and/or based on a comparison of one or more medical device protocols associated with a medical device 126 (stored in a protocol storage 206) and one or more library protocols associated with a protocol library 120, determine a protocol conflict. Such a protocol conflict can comprise a problem that prevents a protocol from being eligible to be transferred from a medical device 126 to the protocol library 120. In this regard, the system 102 can comprise a cloud-based library (e.g., protocol library 120), which can comprise a repository of protocols accessible by a plurality of medical devices 126. In various implementations, the protocol library 120 can comprise a central repository comprising protocols from some or all medical devices 126 of an organization. The medical devices 126 can read or write protocols to the protocol library 120 and/or to their own respective protocol storage 206 (e.g., local to a respective medical device 126). The conflict component 110 can compare one or more aspects of protocols herein. For instance, the conflict component 110 can analyze and compare protocol titles and/or contents of the respective protocols (e.g., defined specifications of the protocols). According to an embodiment, the output component 112 can render and/or generate an output (e.g., an output such as a conflict resolution window viewable or interactable via GUI 122, user device 124, medical device 126, etc.) representative of the protocol conflict determined via the conflict component 110. In this regard, the output component 112 can generate a conflict resolution window (later discussed in greater detail), that can be presented via a user interface, in order to present option(s) for curing a conflict to a user (e.g., a radiologist, nurse, medical imaging technician, etc.)

In various embodiments, the system 102 can utilize hashes to compare protocols herein. Thus, according to an embodiment, the hash component 114 can generate a first hash of a medical device protocol of the one or more medical device protocols and a second hash of a library protocol of the one or more library protocols. It is noted that the hash component 114 can generate hashes of medical device protocols using a defined hash or checksum algorithm. In some embodiments, a defined conflict criterion herein can comprise a match (or non-match) between the first hash and the second hash generated via the hash component 114. For instance, the first hash not matching the second hash can be indicative of a protocol conflict. It is noted, however, that the defined conflict criterion can comprise one or more of a variety of conflict criteria. For example, the defined conflict criterion can additionally, or alternatively, comprise a match between a first title of a medical device protocol of the one or more medical device protocols and a second title of a library protocol of the one or more library protocols. In this regard, a non-match between such protocols can be indicative of a protocol conflict. Additionally, or alternatively, the defined conflict criterion can comprise a quantity of alphanumeric characters represented in a title of a medical device protocol of the one or more medical device protocols comprises greater than a defined maximum number of alphanumeric characters. For example, 64 alphanumeric characters can comprise a defined maximum alphanumeric character limit, though this is purely exemplary. In some embodiments, an alphanumeric character maximum can be based on a capability or limitation of a corresponding medical device 126. In this example, if a protocol title comprises greater than 64 alphanumeric characters, a protocol conflict can be determined to exist (e.g., via the conflict component 110). Additionally, or alternatively, the defined conflict criterion can comprise a presence of a leading space or a trailing space (e.g., a blank space) in a name or title of a medical device protocol of the one or more medical device protocols. In this regard, presence of a leading space or trailing space can result in a conflict being determined to exist (e.g., via the conflict component 110). Additionally, or alternatively, the defined conflict criterion can comprise a first slot number of a medical device protocol of the one or more medical device protocols matching a second slot number of a library protocol of the one or more library protocols. Additionally, or alternatively, the defined conflict criterion can comprise a presence of a duplication of a protocol. It is noted that such a duplication can comprise a first set of parameters or specifications represented in a medical device protocol of the one or more medical device protocols matching a second set of parameters or specifications represented in a library protocol of the one or more library protocols. It is additionally noted that the protocol library can comprise a cloud-based storage communicatively coupled to the medical device 126.

According to an embodiment, the resolution component 118 can, in response to receiving (e.g., via the communication component 116 or GUI 122) selection of conflict resolution determined to resolve the protocol conflict, apply the conflict resolution to a corresponding protocol conflict. Such a conflict resolution can be applied (e.g., by the resolution component 118) prior to pushing the corresponding protocol to the protocol library 120. In various embodiments, the communication component 116 can comprise the hardware required to implement a variety of communication protocols (e.g., infrared (“IR”), shortwave transmission, near-field communication (“NFC”), Bluetooth, Wi-Fi, long-term evolution (“LTE”), 3G, 4G, 5G, 6G, global system for mobile communications (“GSM”), code-division multiple access (“CDMA”), satellite, visual cues, radio waves, etc.) In other embodiments, the system 102 can leverage artificial intelligence and/or machine learning in order to learn to automatically cure a conflict. For instance, the system 102 can, using machine learning, generate a conflict resolution model based on past resolutions of other conflicts, other than an instant conflict. In this regard, in some embodiments, the resolution component 118 can automatically correct a conflict based on a result of an application of an instant conflict to the conflict resolution model.

According to an embodiment, the user device 124 can comprise one or more of a variety of types of user device, such as a computer, smart phone, wearable device, augmented reality (AR) device, virtual reality (VR) device, mixed reality, tablet, camera, PDA, or other suitable user device that can comprise a user interface and/or be communicatively coupled to the system 102. In some embodiments, the system 102 can facilitate a web-based portal or web-based application for a user to access from the user device 124, medical device 126, GUI 122, or another suitable device communicatively coupled to the system 102. In further embodiments, a browser-based interface can serve as a zero footprint, zero download, and/or other universal viewer for the user device 124, medical device 126, GUI 122, or another suitable device communicatively coupled to the system 102. Thus, a user can be enabled, via the system 102 or a communicatively coupled device, to view, edit, and evaluate the protocols through the web-based portal or application. The user interface of the web-based portal/application may be configured to help or guide a user in accessing data and/or functions to facilitate protocol management. In some embodiments, the user interface can be configured according to certain rules, preferences, and/or functions. Furthermore, a user can customize the interface according to respective user preferences and/or requirements.

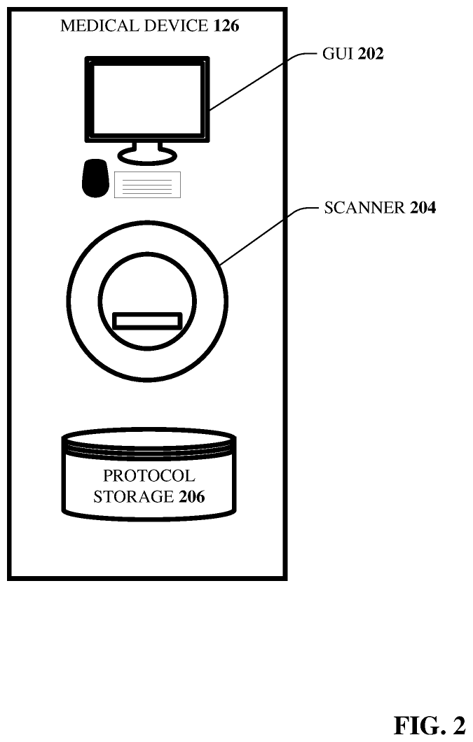

With reference to FIG. 2, it is noted that a medical device 126 (e.g., medical device 126a, medical device 126b, medical device 126c, medical device 126d, and so on) can comprise a GUI 202, scanner 204, and/or protocol storage 206. In this regard, the scanner 204 can be representative of respective MR, CT, x-ray, PET, NM equipment, or other suitable, respective medical imaging equipment that the medical device 126 can utilize. The protocol storage 206 can comprise local protocol storage (e.g., of a medical device 126) on which protocols for the medical device 126 can be stored. Such local medical device protocols can be retrieved from the protocol library 120 and/or generated at the medical device 126. For example, if a hospital or other medical facility acquires a new medical device 126, the protocol storage 206 can be populated with an initial set of protocols from the protocol library 120. Likewise, such local medical device protocols can be pushed to the protocol library 120 so that the protocol can be used on other medical devices in a hospital or medical network.

Various embodiments herein can employ artificial-intelligence or machine learning systems and techniques to facilitate learning user behavior, context-based scenarios, preferences, etc. in order to facilitate taking automated action with high degrees of confidence. Utility-based analysis can be utilized to factor benefit of taking an action against cost of taking an incorrect action. Probabilistic or statistical-based analyses can be employed in connection with the foregoing and/or the following.

It is noted that systems and/or associated controllers, servers, or machine learning components herein can comprise artificial intelligence component(s) which can employ an artificial intelligence (AI) model, a machine learning (ML) model, and/or a deep learning (DL) model that can learn to perform the above or below described functions (e.g., via training using historical training data and/or feedback data).

In some embodiments, system 102 can comprise an AI, ML, and/or DL model that can be trained (e.g., via supervised and/or unsupervised techniques) to perform the above or below-described functions using historical training data comprising various context conditions that correspond to various augmented network optimization operations. In this example, such an AI, ML, and/or DL model can further learn (e.g., via supervised and/or unsupervised techniques) to perform the above or below-described functions using training data comprising feedback data, where such feedback data can be collected and/or stored (e.g., in memory) by the system 102. In this example, such feedback data can comprise the various instructions described above/below that can be input, for instance, to a system herein, over time in response to observed/stored context-based information.

AI/ML/DL components herein can initiate an operation(s) associated with a defined level of confidence determined using information (e.g., feedback data). For example, based on learning to perform such functions described above using feedback data, performance information, and/or past performance information herein, a system 102 herein can initiate an operation associated with determining various thresholds herein (e.g., motion pattern thresholds, input pattern thresholds, similarity thresholds, authentication signal thresholds, audio frequency thresholds, or other suitable thresholds).

In an embodiment, the system 102 can perform a utility-based analysis that factors cost of initiating the above-described operations versus benefit. In this embodiment, the system 102 can use one or more additional context conditions to determine various thresholds herein.

To facilitate the above-described functions, a system 102 herein can perform classifications, correlations, inferences, and/or expressions associated with principles of artificial intelligence. For instance, the system 102 can employ an automatic classification system and/or an automatic classification. In one example, the system 102 can employ a probabilistic and/or statistical-based analysis (e.g., factoring into the analysis utilities and costs) to learn and/or generate inferences. The system 102 can employ any suitable machine-learning based techniques, statistical-based techniques and/or probabilistic-based techniques. For example, the system 102 can employ expert systems, fuzzy logic, support vector machines (SVMs), Hidden Markov Models (HMMs), greedy search algorithms, rule-based systems, Bayesian models (e.g., Bayesian networks), neural networks, other non-linear training techniques, data fusion, utility-based analytical systems, systems employing Bayesian models, and/or the like. In another example, the system 102 can perform a set of machine-learning computations. For instance, the system 102 can perform a set of clustering machine learning computations, a set of logistic regression machine learning computations, a set of decision tree machine learning computations, a set of random forest machine learning computations, a set of regression tree machine learning computations, a set of least square machine learning computations, a set of instance-based machine learning computations, a set of regression machine learning computations, a set of support vector regression machine learning computations, a set of k-means machine learning computations, a set of spectral clustering machine learning computations, a set of rule learning machine learning computations, a set of Bayesian machine learning computations, a set of deep Boltzmann machine computations, a set of deep belief network computations, and/or a set of different machine learning computations.

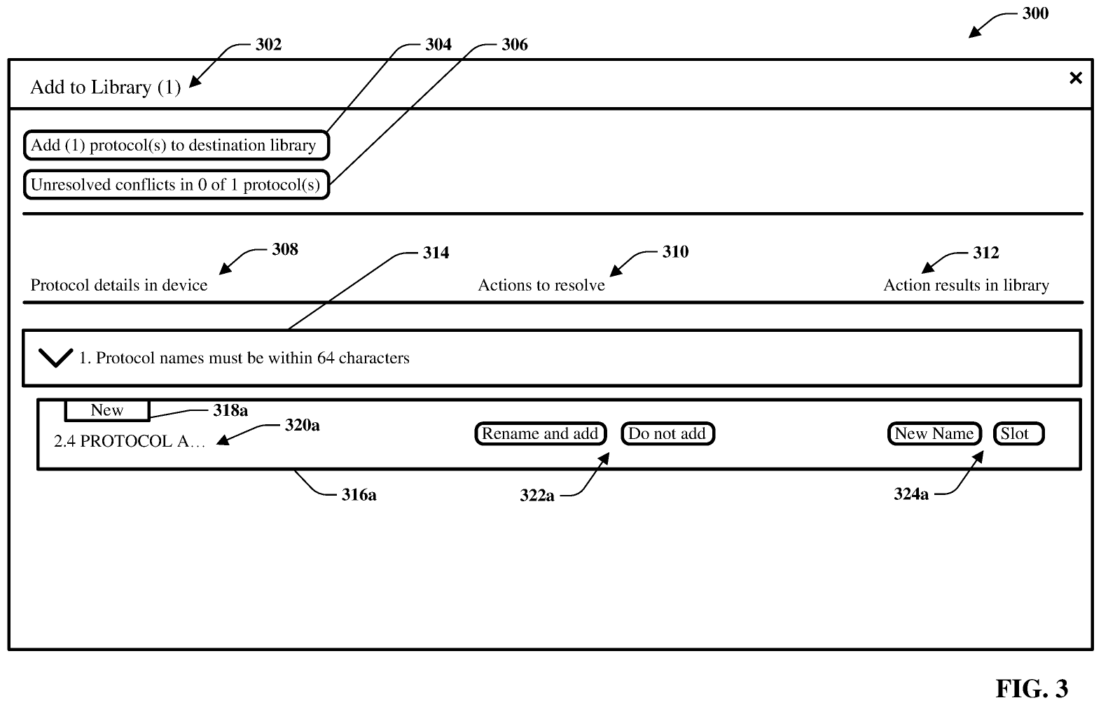

FIGS. 3-7 illustrate various exemplary, nonlimiting conflict resolution windows generated by the system 102 (e.g., via the output component 112) and/or rendered via a UI herein (e.g., GUI 122, user device 124, GUI 202 of the medical device 126, or another suitable output medium).

In FIG. 3, a conflict resolution window 300 is depicted. Conflict resolution window 300 can be generated by the output component 112, and can comprise one or more of a protocol addition quantity 302, destination indicator 304, unresolved conflict indicator 306, protocol details in device column 308, actions to resolve column 310, action results in library column 312, conflict category bar 314, first conflict 316a, label 318a, imaging protocol name 320a, available actions 322a to resolve the first conflict 316a, and/or fields/results 324a. In FIGS. 3-7, the protocol addition quantity 302 can represent the number of protocols that are being pushed to the protocol library 120 from the medical device 126. The destination indicator 304 can represent the destination of protocols (e.g., protocol library 120). The unresolved conflict indicator 306 can represent the number of conflicts that remain unresolved as compared to the number of protocols that are being pushed to the protocol library 120. The protocol details in device column 308 can comprise a column under which details (e.g., slot number, protocol title, etc. of a protocol comprising a conflict or previously comprising a conflict) are depicted in the conflict resolution window. Available actions to resolve a given conflict can be presented in the actions to resolve column 310. Available actions can vary depending on the type of conflict. Such available actions can be presented adjacent to a respective protocol title. Action results in library column 312 can comprise effects and/or available data entry fields associated with a corresponding action from the actions to resolve column 310. In this regard, the effects and/or available data entry fields can be configured to change (e.g., via the output component 112) based upon a selection of an action to resolve a conflict from the actions to resolve column 310. The conflict category bar 314 can comprise a category of conflict under which corresponding conflicts of that category are presented. Label 318 can be indicative of a protocol state of a medical device protocol of one or more medical device protocols. In various embodiments, the label 318 can comprise a defined color based on a type of conflict or other suitable information. For example, the label 318 can be colored orange of there exists a change in a parameter or specification of a protocol (e.g., a deviation). The label 318 can be colored grey if a name of a protocol has changed (e.g., a deviation). It is noted, however, that such colors are purely exemplary and other suitable colors and combinations are envisaged. Likewise, the unresolved conflict indicator 306 can comprise a defined color based on a number of conflicts remaining or other suitable information. For example, the conflict indicator can illuminate green to indicate that no conflicts remain, or red to indicate that there exist outstanding unresolved conflicts. Further, upon resolution of a conflict, the conflict resolution window 300 can comprise a color change representative of resolution of a conflict. For example, upon resolution of the first conflict 316a, contents of the first conflict 316a can be configured to change (e.g., via the output component 112) from red to green, though other suitable color changes can be utilized.

According to an embodiment, conflict resolution window 300 depicts an alphanumeric conflict in which PROTOCOL A (e.g., residing in slot 2.4 of medical device 126) comprises greater than a defined maximum number of characters. In this example, 64 characters is the defined maximum, though this is purely exemplary. In some embodiments, an alphanumeric character maximum can be based on a capability or limitation of a corresponding medical device 126. The conflict resolution window 300 can be configured (e.g., via the output component 112) to present one or more of a variety of available actions to resolve the alphanumeric character conflict. For example, the available actions 322a can comprise one or more of renaming and adding the PROTOCOL A to the protocol library 120, or not adding the PROTOCOL A to the protocol library 120. In this regard, if rename and add is selected, the fields/results 324a can comprise a field at which a new name for PROTOCOL A can be entered and/or a slot number for which to assign the PROTOCOL A in the protocol library 120. It is noted that, in conflict resolution window 300, the label 318a can comprise a label (e.g., a new label) denoting “new” or the like, representative of the medical device protocol existing only on the medical device 126.

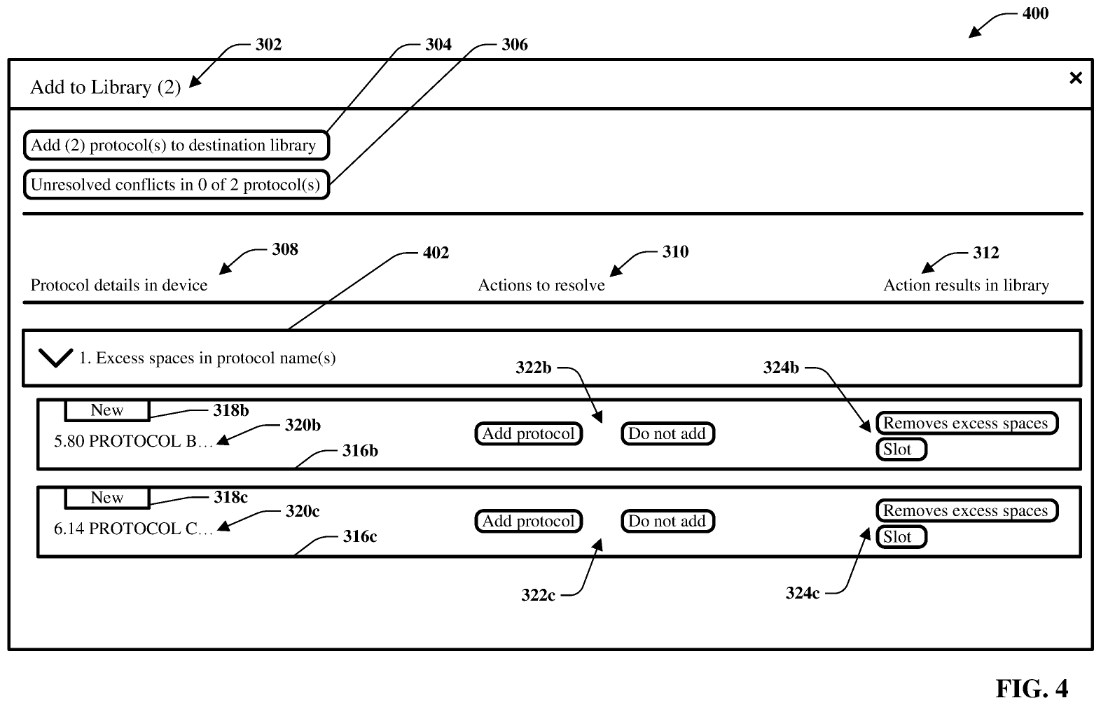

In FIG. 4, a conflict resolution window 400 is depicted. Conflict resolution window 400 can comprise one or more of a protocol addition quantity 302, destination indicator 304, unresolved conflict indicator 306, protocol details in device column 308, actions to resolve column 310, action results in library column 312, conflict category bar 402, first conflict 316b, label 318b, imaging protocol name 320b, available actions 322b to resolve the first conflict 316b, fields/results 324b, second conflict 316c, label 318c, imaging protocol name 320c, available actions 322c to resolve the second conflict 316c, and/or fields/results 324c. According to an embodiment, conflict resolution window 400 depicts a leading or trailing space conflict in which PROTOCOL B (e.g., residing in slot 5.80 of medical device 126) and/or PROTOCOL C (e.g., residing in slot 6.14 of medical device 126) comprise leading or trialing alphanumeric blank spaces (e.g., excess spaces in a protocol name). Avoiding such spaces can be desirable, as some medical devices may not be able to accommodate a protocol title with a leading or trailing space (e.g., based on hardware or software limitations of the respective medical device). The conflict resolution window 400 can be configured (e.g., via the output component 112) to present one or more of a variety of available actions to resolve the leading or trailing space conflict(s). For example, the available actions 322b can comprise one or more of adding the PROTOCOL B to the protocol library 120 (e.g., with leading and trailing spaces automatically removed), or not adding the PROTOCOL B to the protocol library 120. In this regard, if add protocol is selected, the fields/results 324b can comprise the new name for PROTOCOL C (e.g., with leading and trailing spaces automatically removed) and/or a slot number for which to assign the PROTOCOL C in the protocol library 120. Likewise, the available actions 322c can comprise one or more of adding the PROTOCOL C to the protocol library 120 (e.g., with leading and trailing spaces automatically removed), or not adding the PROTOCOL C to the protocol library 120. In this regard, if add protocol is selected, the fields/results 324c can comprise the new name for PROTOCOL C (e.g., with leading and trailing spaces automatically removed) and/or a slot number for which to assign the PROTOCOL C in the protocol library 120.

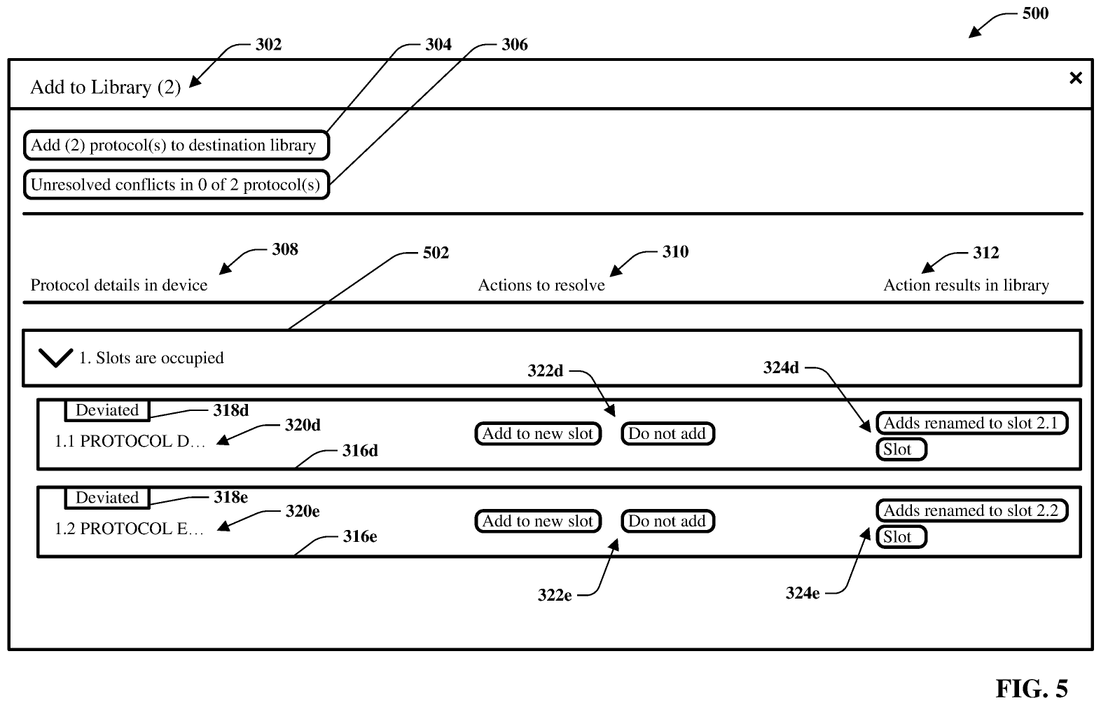

In FIG. 5, a conflict resolution window 500 is depicted. Conflict resolution window 500 can comprise one or more of a protocol addition quantity 302, destination indicator 304, unresolved conflict indicator 306, protocol details in device column 308, actions to resolve column 310, action results in library column 312, conflict category bar 502, first conflict 316d, label 318d, imaging protocol name 320d, available actions 322d to resolve the first conflict 316d, fields/results 324d, second conflict 316c, label 318e, imaging protocol name 320e, available actions 322e to resolve the second conflict 316e, and/or fields/results 324c. According to an embodiment, conflict resolution window 500 depicts a slot number conflict in which slot numbers of PROTOCOL D (e.g., residing in slot 1.1 of medical device 126) and/or slot numbers of PROTOCOL E (e.g., residing in slot 1.2 of medical device 126) are already occupied in the protocol library 120. In this regard, absent conflict resolution, pushing PROTOCOL D and/or PROTOCOL E to the protocol library 120 can result in an unintentional overwriting of protocols in the protocol library 120. The conflict resolution window 500 can be configured (e.g., via the output component 112) to present one or more of a variety of available actions to resolve the slot number conflict(s). For example, the available actions 322d can comprise one or more of adding the PROTOCOL D to the protocol library 120 (e.g., with a new slot number not currently occupied in the protocol library 120), or not adding the PROTOCOL D to the protocol library 120. In this regard, if add protocol is selected, the fields/results 324d can comprise the new slot number for PROTOCOL D. Likewise, the available actions 322c can comprise one or more of adding the PROTOCOL E to the protocol library 120 (e.g., with a new slot number not currently occupied in the protocol library 120), or not adding the PROTOCOL E to the protocol library 120. In this regard, if add protocol is selected, the fields/results 324e can comprise the new slot number for PROTOCOL E. It is noted that, in conflict resolution window 500, the label 318d and/or label 318e can comprise a label (e.g., a deviated label) denoting “deviated” or the like, representative of a medical device protocol of the one or more medical device protocols comprising a slot number that deviates from a slot number of a library protocol of the one or more library protocols.

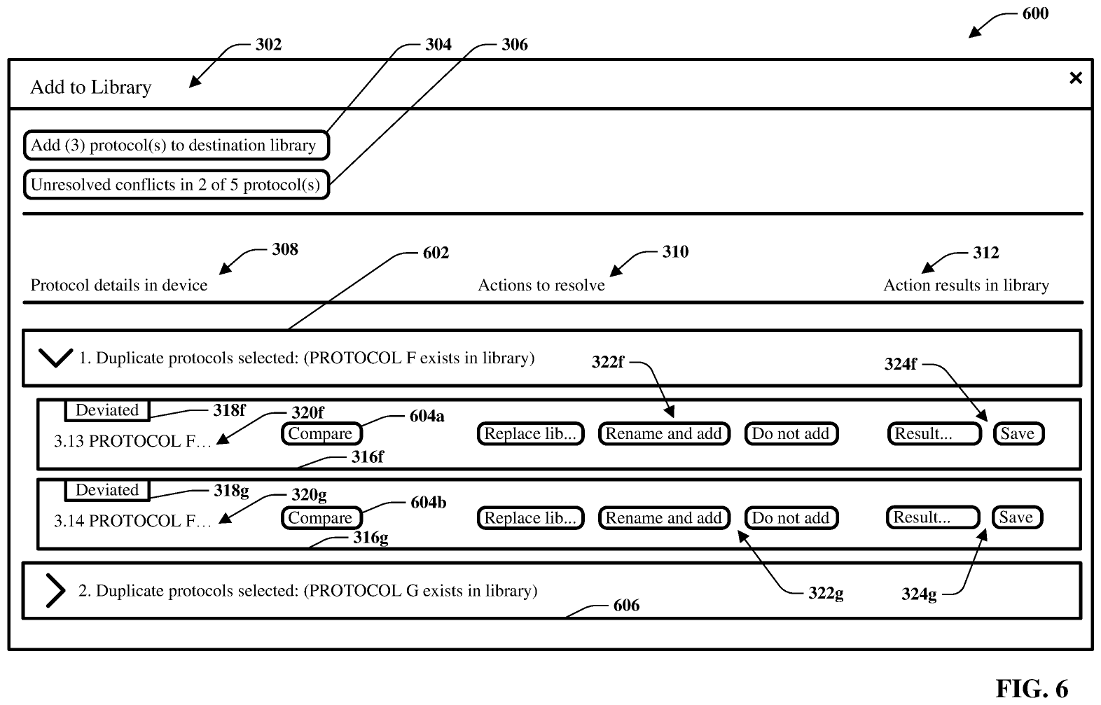

In FIG. 6, a conflict resolution window 600 is depicted. Conflict resolution window 600 can comprise one or more of a protocol addition quantity 302, destination indicator 304, unresolved conflict indicator 306, protocol details in device column 308, actions to resolve column 310, action results in library column 312, first conflict category bar 602, first conflict 316f, label 318f, imaging protocol name 320f, available actions 322f to resolve the first conflict 316f, fields/results 324f, compare button 604a, second conflict 316g, label 318g, imaging protocol name 320g, available actions 322g to resolve the second conflict 316g, fields/results 324g, compare button 604b, and/or second conflict category bar 606. According to an embodiment, conflict resolution window 600 depicts a duplicate protocol conflict in the title of PROTOCOL F already exists in the protocol library 120. In this regard, absent conflict resolution, pushing PROTOCOL F to the protocol library can result in an unintentional overwriting of protocols in the protocol library 120. The conflict resolution window 600 can be configured (e.g., via the output component 112) to present one or more of a variety of available actions to resolve the duplicate protocol conflict(s). For example, the available actions 322f can comprise one or more of adding the PROTOCOL F to the protocol library (e.g., replacing by overwriting the corresponding protocol in the protocol library), renaming and adding the PROTOCOL F to the protocol library, and/or not adding the PROTOCOL F to the protocol library 120. In this regard, if the protocol is added but not renamed, the fields/results 324f can comprise a slot number selection for PROTOCOL F. If rename and add PROTOCOL F is selected, the fields/results 324f can comprise a new name and slot number for PROTOCOL F. Likewise, the available actions 322g can comprise one or more of adding the PROTOCOL F to the protocol library (e.g., replacing by overwriting the corresponding protocol in the protocol library), renaming and adding the PROTOCOL F to the protocol library, and/or not adding the PROTOCOL F to the protocol library 120. In this regard, if the protocol is added but not renamed, the fields/results 324g can comprise a slot number selection for PROTOCOL F. If rename and add PROTOCOL F is selected, the fields/results 324gcan comprise a new name and slot number for PROTOCOL F. It is noted that, in conflict resolution window 600, the label 318f and/or label 318g can comprise a label (e.g., a deviated label) denoting “deviated” or the like, representative of a medical device protocol of the one or more medical device protocols comprising a name that deviates from a name of a library protocol of the one or more library protocols. It is noted that conflict types can be configured (e.g., via the output component 112) to be presented in a defined order. In this regard, first conflict category bar 602 can be configured to be presented (e.g., via the output component 112) above second conflict category bar 606. For example, a hierarchy of conflict type sequencing can comprise duplicate protocol name, then duplicate protocol contents, then alphanumeric character or length conflicts, though this sequencing is purely exemplary and other suitable sequences are envisaged.

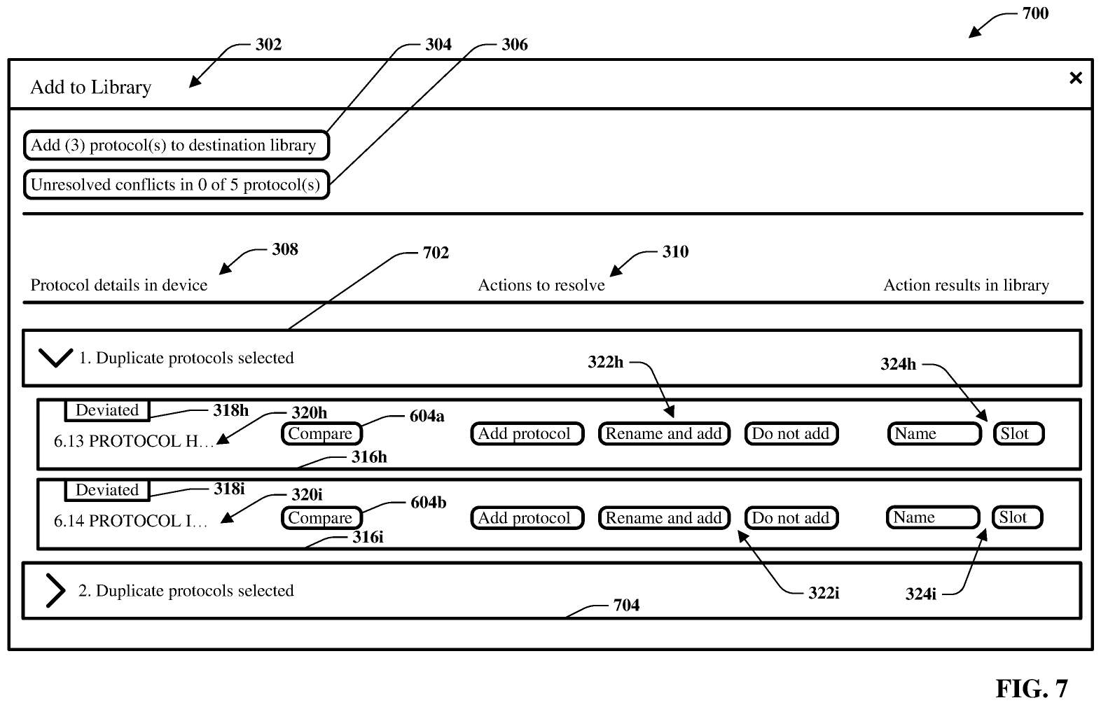

In FIG. 7, a conflict resolution window 700 is depicted. Conflict resolution window 700 can comprise one or more of a protocol addition quantity 302, destination indicator 304, unresolved conflict indicator 306, protocol details in device column 308, actions to resolve column 310, action results in library column 312, first conflict category bar 702, first conflict 316h, label 318h, imaging protocol name 320h, available actions 322h to resolve the first conflict 316h, fields/results 324h, compare button 604a, second conflict 316i, label 318i, imaging protocol name 320i, available actions 322i to resolve the second conflict 316i, fields/results 324i, compare button 604b, and/or second conflict category bar 704. According to an embodiment, conflict resolution window 700 depicts a duplicate protocol conflict in which the parameters or specifications of PROTOCOL H and/or PROTOCOL I already exist in a differently titled protocol in the protocol library 120. In this regard, absent conflict resolution, pushing PROTOCOL H or PROTOCOL I to the protocol library can result in redundant protocols in the protocol library 120. The conflict resolution window 700 can be configured (e.g., via the output component 112) to present one or more of a variety of available actions to resolve the duplicate protocol conflict(s). For example, the available actions 322h can comprise one or more of adding the PROTOCOL H to the protocol library, renaming and adding the PROTOCOL H to the protocol library, and/or not adding the PROTOCOL H to the protocol library 120. In this regard, if the protocol is added, the fields/results 324f can comprise a slot number selection for PROTOCOL H. If rename and add PROTOCOL H is selected, the fields/results 324h can comprise a new name and slot number for PROTOCOL H. Likewise, the available actions 322i can comprise one or more of adding the PROTOCOL I to the protocol library, renaming and adding the PROTOCOL I to the protocol library, and/or not adding the PROTOCOL I to the protocol library 120. In this regard, if the protocol is added, the fields/results 324i can comprise a slot number selection for PROTOCOL I. If rename and add PROTOCOL I is selected, the fields/results 324i can comprise a new name and slot number for PROTOCOL I. It is noted that, in conflict resolution window 700, the label 318h and/or label 318i can comprise a label (e.g., a deviated label) denoting “deviated” or the like, representative of a medical device protocol of the one or more medical device protocols comprising one or more parameters or specifications that deviate from one or more parameters or specifications of a library protocol of the one or more library protocols.

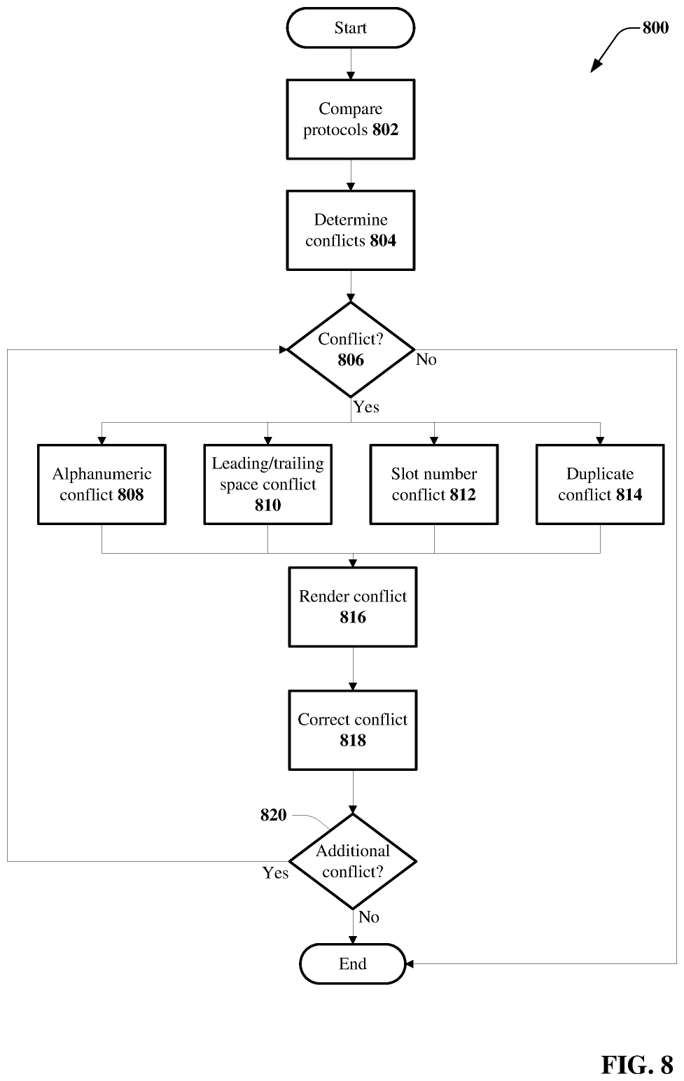

FIG. 8 depicts a flowchart of a process associated with imaging protocol conflict management in accordance with one or more embodiments described herein. At 802, a comparison of one or more medical device protocols associated with a medical device and one or more library protocols associated with a protocol library can be executed (e.g., via the conflict component 110). At 804, a protocol conflict can be determined (e.g., via the conflict component 110) based on the comparison at 802 and according to a defined conflict criterion (e.g., match or non-match between a first hash and a second hash generated via the hash component 114, a match between a first title of a medical device protocol of the one or more medical device protocols and a second title of a library protocol of the one or more library protocols, a quantity of alphanumeric characters represented in a title of a medical device protocol of the one or more medical device protocols comprises greater than a defined maximum number of alphanumeric characters, presence of a leading space or a trailing space (e.g., a blank space) in a name or title of a medical device protocol of the one or more medical device protocols, a first slot number of a medical device protocol of the one or more medical device protocols matching a second slot number of a library protocol of the one or more library protocols, duplication of a protocol, etc.) At 806, if no conflict exists, the process 800 can end or start over. However, if at 806, a conflict exists, the conflict can be categorized (e.g., via the conflict component 110) according to a type of conflict. Such types of conflicts can comprise an alphanumeric conflict at 808, a leading/trailing space conflict at 810, a slot number conflict at 812, and/or a duplicate conflict 814. At 816, an output representative of the protocol conflict can be generated and/or rendered (e.g., by the output component 112) (e.g., via GUI 122, GUI 202, user device 124, or another suitable output medium). In this regard, the output component 112 can render a graphical user interface, comprising conflict resolution window representative of the protocol conflict, via the medical device 126. At 818, based upon receiving an input corresponding to the protocol conflict, to correct the protocol conflict (e.g., via the GUI 122, user device 124, GUI 202, and/or communication component 116), the conflict can be corrected (e.g., via the resolution component 118). At 820, if there exist any outstanding protocol conflicts, the process can return to 806 for further processing and/or correction. However, if at 820, no outstanding protocol conflicts exist, the process 800 can end or start over.

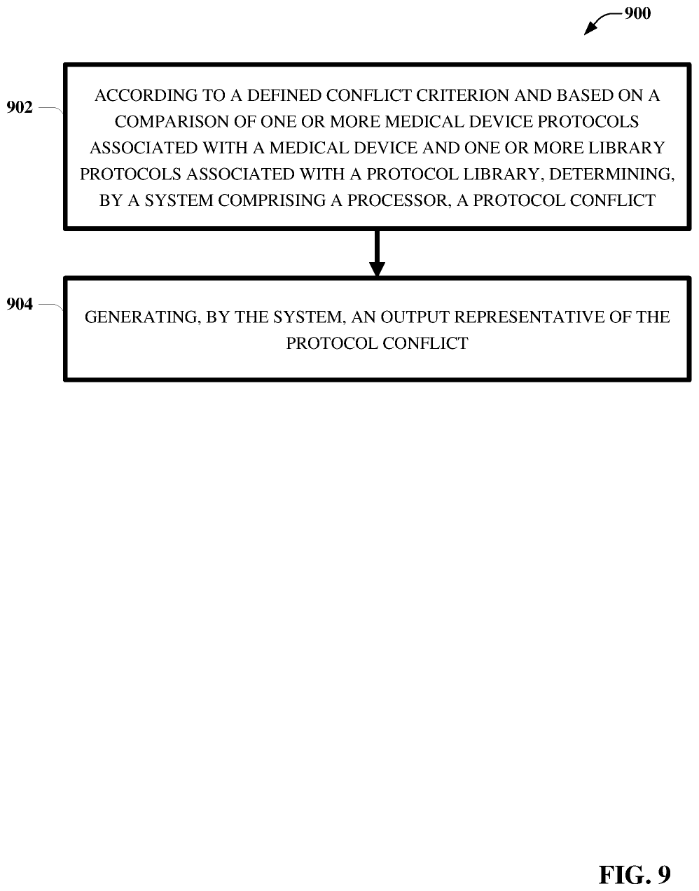

FIG. 9 illustrates a block flow diagram for a process associated with imaging protocol conflict management in accordance with one or more embodiments described herein. At 902, the process 900 can comprise, according to a defined conflict criterion and based on a comparison of one or more medical device protocols associated with a medical device and one or more library protocols associated with a protocol library, determining (e.g., via the conflict component 110), by a system (e.g., system 102) comprising a processor (e.g., processor 106), a protocol conflict. At 904, the process 900 can comprise generating (e.g., via the output component 112), by the system (e.g., system 102), an output representative of the protocol conflict.

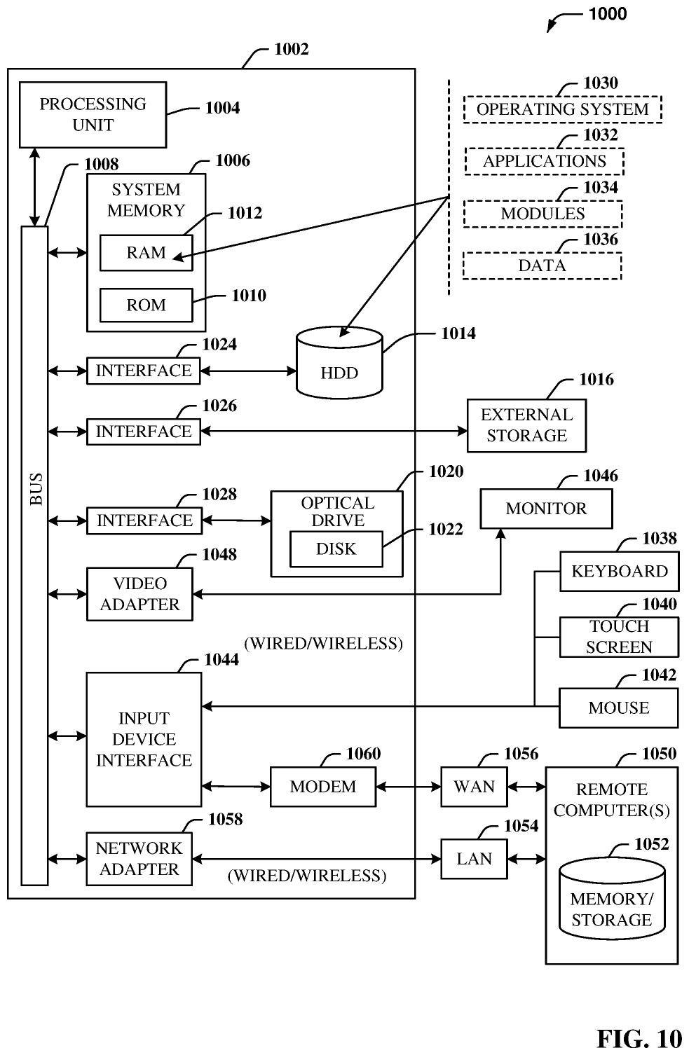

In order to provide additional context for various embodiments described herein, FIG. 10 and the following discussion are intended to provide a brief, general description of a suitable computing environment 1000 in which the various embodiments of the embodiment described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the various methods can be practiced with other computer system configurations, including single-processor or multiprocessor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data, or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory, or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries, or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared, and other wireless media.

With reference again to FIG. 10, the example environment 1000 for implementing various embodiments of the aspects described herein includes a computer 1002, the computer 1002 including a processing unit 1004, a system memory 1006 and a system bus 1008. The system bus 1008 couples system components including, but not limited to, the system memory 1006 to the processing unit 1004. The processing unit 1004 can be any of various commercially available processors. Dual microprocessors and other multi-processor architectures can also be employed as the processing unit 1004.

The system bus 1008 can be any of several types of bus structure that can further interconnect to a memory bus (with or without a memory controller), a peripheral bus, and a local bus using any of a variety of commercially available bus architectures. The system memory 1006 includes ROM 1010 and RAM 1012. A basic input/output system (BIOS) can be stored in a non-volatile memory such as ROM, erasable programmable read only memory (EPROM), EEPROM, where BIOS contains the basic routines that help transfer information between elements within the computer 1002, such as during startup. The RAM 1012 can also include a high-speed RAM such as static RAM for caching data.

The computer 1002 further includes an internal hard disk drive (HDD) 1014 (e.g., EIDE, SATA), one or more external storage devices 1016 (e.g., a magnetic floppy disk drive (FDD) 1016, a memory stick or flash drive reader, a memory card reader, etc.) and an optical disk drive 1020 (e.g., which can read or write from a CD-ROM disc, a DVD, a BD, etc.). While the internal HDD 1014 is illustrated as located within the computer 1002, the internal HDD 1014 can also be configured for external use in a suitable chassis (not shown). Additionally, while not shown in environment 1000, a solid-state drive (SSD) could be used in addition to, or in place of, an HDD 1014. The HDD 1014, external storage device(s) 1016 and optical disk drive 1020 can be connected to the system bus 1008 by an HDD interface 1024, an external storage interface 1026 and an optical drive interface 1028, respectively. The interface 1024 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1002, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1012, including an operating system 1030, one or more application programs 1032, other program modules 1034 and program data 1036. All or portions of the operating system, applications, modules, and/or data can also be cached in the RAM 1012. The systems and methods described herein can be implemented utilizing various commercially available operating systems or combinations of operating systems.

Computer 1002 can optionally comprise emulation technologies. For example, a hypervisor (not shown) or other intermediary can emulate a hardware environment for operating system 1030, and the emulated hardware can optionally be different from the hardware illustrated in FIG. 10. In such an embodiment, operating system 1030 can comprise one virtual machine (VM) of multiple VMs hosted at computer 1002. Furthermore, operating system 1030 can provide runtime environments, such as the Java runtime environment or the .NET framework, for applications 1032. Runtime environments are consistent execution environments that allow applications 1032 to run on any operating system that includes the runtime environment. Similarly, operating system 1030 can support containers, and applications 1032 can be in the form of containers, which are lightweight, standalone, executable packages of software that include, e.g., code, runtime, system tools, system libraries and settings for an application.

Further, computer 1002 can be enabled with a security module, such as a trusted processing module (TPM). For instance, with a TPM, boot components hash next in time boot components, and wait for a match of results to secured values, before loading a next boot component. This process can take place at any layer in the code execution stack of computer 1002, e.g., applied at the application execution level or at the operating system (OS) kernel level, thereby enabling security at any level of code execution.

A user can enter commands and information into the computer 1002 through one or more wired/wireless input devices, e.g., a keyboard 1038, a touch screen 1040, and a pointing device, such as a mouse 1042. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1004 through an input device interface 1044 that can be coupled to the system bus 1008, but can be connected by other interfaces, such as a parallel port, an IEEE 1394 serial port, a game port, a USB port, an IR interface, a BLUETOOTH® interface, etc.

A monitor 1046 or other type of display device can be also connected to the system bus 1008 via an interface, such as a video adapter 1048. In addition to the monitor 1046, a computer typically includes other peripheral output devices (not shown), such as speakers, printers, etc.

The computer 1002 can operate in a networked environment using logical connections via wired and/or wireless communications to one or more remote computers, such as a remote computer(s) 1050. The remote computer(s) 1050 can be a workstation, a server computer, a router, a personal computer, portable computer, microprocessor-based entertainment appliance, a peer device or other common network node, and typically includes many or all of the elements described relative to the computer 1002, although, for purposes of brevity, only a memory/storage device 1052 is illustrated. The logical connections depicted include wired/wireless connectivity to a local area network (LAN) 1054 and/or larger networks, e.g., a wide area network (WAN) 1056. Such LAN and WAN networking environments are commonplace in offices and companies, and facilitate enterprise-wide computer networks, such as intranets, all of which can connect to a global communications network, e.g., the Internet.

When used in a LAN networking environment, the computer 1002 can be connected to the local network 1054 through a wired and/or wireless communication network interface or adapter 1058. The adapter 1058 can facilitate wired or wireless communication to the LAN 1054, which can also include a wireless access point (AP) disposed thereon for communicating with the adapter 1058 in a wireless mode.

When used in a WAN networking environment, the computer 1002 can include a modem 1060 or can be connected to a communications server on the WAN 1056 via other means for establishing communications over the WAN 1056, such as by way of the Internet. The modem 1060, which can be internal or external and a wired or wireless device, can be connected to the system bus 1008 via the input device interface 1044. In a networked environment, program modules depicted relative to the computer 1002 or portions thereof, can be stored in the remote memory/storage device 1052. It will be appreciated that the network connections shown are example and other means of establishing a communications link between the computers can be used.

When used in either a LAN or WAN networking environment, the computer 1002 can access cloud storage systems or other network-based storage systems in addition to, or in place of, external storage devices 1016 as described above. Generally, a connection between the computer 1002 and a cloud storage system can be established over a LAN 1054 or WAN 1056 e.g., by the adapter 1058 or modem 1060, respectively. Upon connecting the computer 1002 to an associated cloud storage system, the external storage interface 1026 can, with the aid of the adapter 1058 and/or modem 1060, manage storage provided by the cloud storage system as it would other types of external storage. For instance, the external storage interface 1026 can be configured to provide access to cloud storage sources as if those sources were physically connected to the computer 1002.

The computer 1002 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

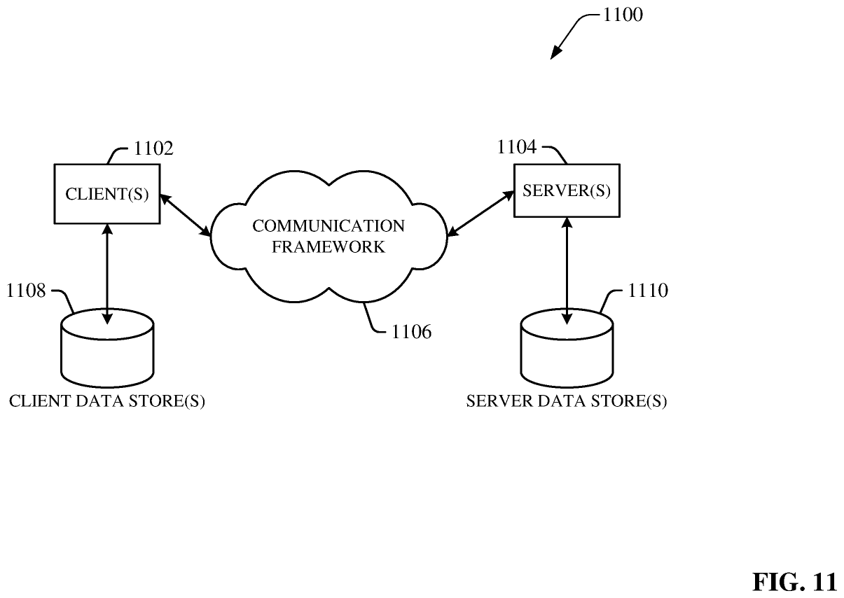

Referring now to FIG. 11, there is illustrated a schematic block diagram of a computing environment 1100 in accordance with this specification. The system 1100 includes one or more client(s) 1102, (e.g., computers, smart phones, tablets, cameras, PDA's). The client(s) 1102 can be hardware and/or software (e.g., threads, processes, computing devices). The client(s) 1102 can house cookie(s) and/or associated contextual information by employing the specification, for example.

The system 1100 also includes one or more server(s) 1104. The server(s) 1104 can also be hardware or hardware in combination with software (e.g., threads, processes, computing devices). The servers 1104 can house threads to perform transformations of media items by employing aspects of this disclosure, for example. One possible communication between a client 1102 and a server 1104 can be in the form of a data packet adapted to be transmitted between two or more computer processes wherein data packets may include coded analyzed headspaces and/or input. The data packet can include a cookie and/or associated contextual information, for example. The system 1100 includes a communication framework 1106 (e.g., a global communication network such as the Internet) that can be employed to facilitate communications between the client(s) 1102 and the server(s) 1104.

Communications can be facilitated via a wired (including optical fiber) and/or wireless technology. The client(s) 1102 are operatively connected to one or more client data store(s) 1108 that can be employed to store information local to the client(s) 1102 (e.g., cookie(s) and/or associated contextual information). Similarly, the server(s) 1104 are operatively connected to one or more server data store(s) 1110 that can be employed to store information local to the servers 1104.

In one exemplary implementation, a client 1102 can transfer an encoded file, (e.g., encoded media item), to server 1104. Server 1104 can store the file, decode the file, or transmit the file to another client 1102. It is noted that a client 1102 can also transfer uncompressed file to a server 1104 and server 1104 can compress the file and/or transform the file in accordance with this disclosure. Likewise, server 1104 can encode information and transmit the information via communication framework 1106 to one or more clients 1102.

The illustrated aspects of the disclosure may also be practiced in distributed computing environments where certain tasks are performed by remote processing devices that are linked through a communications network. In a distributed computing environment, program modules can be located in both local and remote memory storage devices.

The above description includes non-limiting examples of the various embodiments. It is, of course, not possible to describe every conceivable combination of components or methods for purposes of describing the disclosed subject matter, and one skilled in the art may recognize that further combinations and permutations of the various embodiments are possible. The disclosed subject matter is intended to embrace all such alterations, modifications, and variations that fall within the spirit and scope of the appended claims.

With regard to the various functions performed by the above-described components, devices, circuits, systems, etc., the terms (including a reference to a “means”) used to describe such components are intended to also include, unless otherwise indicated, any structure(s) which performs the specified function of the described component (e.g., a functional equivalent), even if not structurally equivalent to the disclosed structure. In addition, while a particular feature of the disclosed subject matter may have been disclosed with respect to only one of several implementations, such feature may be combined with one or more other features of the other implementations as may be desired and advantageous for any given or particular application.

The terms “exemplary” and/or “demonstrative” as used herein are intended to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent structures and techniques known to one skilled in the art. Furthermore, to the extent that the terms “includes,” “has,” “contains,” and other similar words are used in either the detailed description or the claims, such terms are intended to be inclusive-in a manner similar to the term “comprising” as an open transition word-without precluding any additional or other elements.

The term “or” as used herein is intended to mean an inclusive “or” rather than an exclusive “or.” For example, the phrase “A or B” is intended to include instances of A, B, and both A and B. Additionally, the articles “a” and “an” as used in this application and the appended claims should generally be construed to mean “one or more” unless either otherwise specified or clear from the context to be directed to a singular form.

The term “set” as employed herein excludes the empty set, i.e., the set with no elements therein. Thus, a “set” in the subject disclosure includes one or more elements or entities. Likewise, the term “group” as utilized herein refers to a collection of one or more entities.

The description of illustrated embodiments of the subject disclosure as provided herein, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as one skilled in the art can recognize. In this regard, while the subject matter has been described herein in connection with various embodiments and corresponding drawings, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

Further aspects of various embodiments described herein are provided by the subject matter of the following clauses:

1. A system, comprising:
a memory that stores computer executable components; and
a processor that executes the computer executable components stored in the memory, wherein the computer executable components comprise:
a conflict component that, according to a defined conflict criterion and based on a comparison of one or more medical device protocols associated with a medical device and one or more library protocols associated with a protocol library, determines a protocol conflict; and
an output component that renders an output representative of the protocol conflict.

2. The system of any preceding clause, wherein the computer executable components further comprise:
a hash component that generates a first hash of a medical device protocol of the one or more medical device protocols and a second hash of a library protocol of the one or more library protocols.

3. The system of any preceding clause, wherein the defined conflict criterion comprises a match between the first hash and the second hash.

4. The system of any preceding clause, wherein the defined conflict criterion comprises a match between a first title of a medical device protocol of the one or more medical device protocols and a second title of a library protocol of the one or more library protocols.

5. The system of any preceding clause, wherein the defined conflict criterion comprises a quantity of alphanumeric characters represented in a title of a medical device protocol of the one or more medical device protocols.

6. The system of any preceding clause, wherein the defined conflict criterion comprises a presence of a leading space or a trailing space in a name of a title of a medical device protocol of the one or more medical device protocols.

7. The system of any preceding clause, wherein the defined conflict criterion comprises a first slot number of a medical device protocol of the one or more medical device protocols matching a second slot number of a library protocol of the one or more library protocols.

8. The system of any preceding clause, wherein the defined conflict criterion comprises a presence of a duplication of a protocol.

9. The system of any preceding clause, wherein the duplication comprises a first set of parameters represented in a medical device protocol of the one or more medical device protocols matching a second set of parameters represented in a library protocol of the one or more library protocols.

10. The system of any preceding clause, wherein the computer executable components further comprise:
a resolution component that, in response to receiving selection of conflict resolution determined to resolve the protocol conflict, applies the conflict resolution to the protocol conflict.

11. The system of any preceding clause, wherein the protocol library is stored in a cloud-based storage communicatively coupled to the medical device.

12. The system of clause 1 above with any set of combinations of systems 2-11 above. 13. A method, comprising:
according to a defined conflict criterion and based on a comparison of one or more medical device protocols associated with a medical device and one or more library protocols associated with a protocol library, determining, by a system comprising a processor, a protocol conflict; and
generating, by the system, an output representative of the protocol conflict.

14. The method of any preceding clause, further comprising: rendering, by the system, a graphical user interface, comprising conflict resolution window representative of the protocol conflict, via the medical device.

15. The method of any preceding clause, wherein the conflict resolution window comprises a label indicative of a protocol state of a medical device protocol of the one or more medical device protocols.

16. The method of any preceding clause, wherein the label comprises a new label representative of the medical device protocol existing only on the medical device.

17. The method of any preceding clause, wherein the label comprises a deviated label representative of a medical device protocol of the one or more medical device protocols comprising one or more parameters that deviate from one or more parameters of a library protocol of the one or more library protocols.

18. The method of any preceding clause, wherein the label comprises a deviated label representative of a medical device protocol of the one or more medical device protocols comprising a name that deviates from a name of a library protocol of the one or more library protocols.

19. The method of any preceding clause, wherein the label comprises a deviated label representative of a medical device protocol of the one or more medical device protocols comprising a slot number that deviates from a slot number of a library protocol of the one or more library protocols.

20. The method of clause 13 above with any set of combinations of methods 14-19 above.

21. A non-transitory machine-readable storage medium, comprising executable instructions that, when executed by a processor, facilitate performance of operations, comprising:
according to a defined conflict criterion and based on a comparison of one or more medical device protocols associated with a medical device and one or more library protocols associated with a protocol library, determining a protocol conflict; and
rendering an output representative of the protocol conflict via a user interface of the medical device.

22. The non-transitory machine-readable storage medium of any preceding clause, wherein the medical device comprises x-ray machine, computed tomography scanner, or a magnetic resonance imaging machine.

23. The non-transitory machine-readable storage medium of clause 21 above with any set of combinations of non-transitory machine-readable storage mediums 22 above.

1. A system, comprising:
a memory that stores computer executable components; and
a processor that executes the computer executable components stored in the memory, wherein the computer executable components comprise:
a conflict component that, according to a defined conflict criterion and based on a comparison of one or more medical device protocols associated with a medical device and one or more library protocols associated with a protocol library, determines a protocol conflict; and
an output component that renders an output representative of the protocol conflict.

2. The system of claim 1, wherein the computer executable components further comprise:
a hash component that generates a first hash of a medical device protocol of the one or more medical device protocols and a second hash of a library protocol of the one or more library protocols.

3. The system of claim 2, wherein the defined conflict criterion comprises a match between the first hash and the second hash.

4. The system of claim 1, wherein the defined conflict criterion comprises a match between a first title of a medical device protocol of the one or more medical device protocols and a second title of a library protocol of the one or more library protocols.

5. The system of claim 1, wherein the defined conflict criterion comprises a quantity of alphanumeric characters represented in a title of a medical device protocol of the one or more medical device protocols.

6. The system of claim 1, wherein the defined conflict criterion comprises a presence of a leading space or a trailing space in a name of a title of a medical device protocol of the one or more medical device protocols.

7. The system of claim 1, wherein the defined conflict criterion comprises a first slot number of a medical device protocol of the one or more medical device protocols matching a second slot number of a library protocol of the one or more library protocols.

8. The system of claim 1, wherein the defined conflict criterion comprises a presence of a duplication of a protocol.

9. The system of claim 8, wherein the duplication comprises a first set of parameters represented in a medical device protocol of the one or more medical device protocols matching a second set of parameters represented in a library protocol of the one or more library protocols.

10. The system of claim 1, wherein the computer executable components further comprise:
a resolution component that, in response to receiving selection of conflict resolution determined to resolve the protocol conflict, applies the conflict resolution to the protocol conflict.

11. The system of claim 1, wherein the protocol library is stored in a cloud-based storage communicatively coupled to the medical device.

12. A method, comprising:
according to a defined conflict criterion and based on a comparison of one or more medical device protocols associated with a medical device and one or more library protocols associated with a protocol library, determining, by a system comprising a processor, a protocol conflict; and
generating, by the system, an output representative of the protocol conflict.

13. The method of claim 12, further comprising:
rendering, by the system, a graphical user interface, comprising conflict resolution window representative of the protocol conflict, via the medical device.

14. The method of claim 13, wherein the conflict resolution window comprises a label indicative of a protocol state of a medical device protocol of the one or more medical device protocols.

15. The method of claim 14, wherein the label comprises a new label representative of the medical device protocol existing only on the medical device.

16. The method of claim 14, wherein the label comprises a deviated label representative of a medical device protocol of the one or more medical device protocols comprising one or more parameters that deviate from one or more parameters of a library protocol of the one or more library protocols.

17. The method of claim 14, wherein the label comprises a deviated label representative of a medical device protocol of the one or more medical device protocols comprising a name that deviates from a name of a library protocol of the one or more library protocols.

18. The method of claim 14, wherein the label comprises a deviated label representative of a medical device protocol of the one or more medical device protocols comprising a slot number that deviates from a slot number of a library protocol of the one or more library protocols.

19. A non-transitory machine-readable storage medium, comprising executable instructions that, when executed by a processor, facilitate performance of operations, comprising:
according to a defined conflict criterion and based on a comparison of one or more medical device protocols associated with a medical device and one or more library protocols associated with a protocol library, determining a protocol conflict; and
rendering an output representative of the protocol conflict via a user interface of the medical device.

20. The non-transitory machine-readable storage medium of claim 19, wherein the medical device comprises x-ray machine, computed tomography scanner, or a magnetic resonance imaging machine.
